(19) United States
(12) Patent Application Publication
(10) Pub. No.: US 2026/0192090 A1
(43) Pub. Date: Jul. 9, 2026

(54) CATHETER HUB WITH ENHANCED STRAIN RELIEF CAPABILITY AND RELATED METHODS

(71) Applicant: BARD PERIPHERAL VASCULAR, INC., Franklin Lakes, NJ (US)

(72) Inventor: Casey Rockwood, Jenks, OK (US)

(21) Appl. No.: 19/130,870

(22) PCT Filed: Nov. 16, 2022
(86) PCT No.: PCT/US2022/050094

§ 371 Date: May 16, 2025

(51) Int. Cl. A61M 25/00; A61M 25/09; A61M 25/10

(52) U.S. Cl. CPC A61M 25/0097; A61M 2025/0098; A61M 25/09; A61M 2025/1061

(57) ABSTRACT

A catheter hub (12) provides enhanced strain relief for a catheter shaft (14). The hub includes a body (18) adapted for connecting to the catheter shaft. The body has a plurality of circumferentially spaced, elongated strain relief members (30) formed of a material at least as rigid as a material of the body, and which may be tapered in a longitudinal direction to provide variable rigidity. A method of manufacturing a catheter hub includes attaching the plurality of strain relief members to the hub body, such as by overmolding.

### BACKGROUND

Typical balloon catheters include a hub connected to a proximal end of a shaft carrying an inflatable balloon along a distal end for performing a medical procedure, such as percutaneous transluminal angioplasty (PTA). A strain relief member may be provided over the catheter shaft to provide support and prevent kinking of the shaft/guidewire lumen at the hub. The strain relief member is usually made of silicone rubber to afford flexibility while providing support to the shaft.

The use of an additional element composed of a material different from that of the hub or the shaft increases the number of components required for assembling the catheter. This also increases manufacturing expense and complexity, resulting in a higher cost. Using silicone rubber also makes it difficult to vary the rigidity of the strain relief element significantly as it extends in the longitudinal direction from the hub.

Accordingly, a need is identified for hub for a catheter that reduces the number of components formed of a material different from the hub to provide strain relief, and also facilitates varying the flexibility, and hence the degree of strain relief provided. A reduction in manufacturing complexity and expense could also thus be realized by using a single material to form the hub and to provide strain relief.

### SUMMARY

An object of the disclosure is to provide a catheter hub with enhanced strain relief. This may be achieved by providing circumferentially spaced, elongated strain relief members of varying rigidity in a longitudinal direction for engaging the catheter shaft when connected to the hub. The strain relief members may be formed of the same material as the body of the hub, such as by being an integral part thereof. A reduction in manufacturing complexity and expense would thus be realized.

According to a first aspect of the disclosure, a catheter hub provides enhanced strain relief for a catheter shaft. The hub includes a body adapted for connecting to the catheter shaft, the body including a plurality of circumferentially spaced, elongated strain relief members formed of a material at least as rigid as a material of the body.

In one embodiment, the body includes a first port for supplying an inflation fluid to the catheter shaft and a second port for introducing a guidewire to the catheter shaft. The plurality of strain relief members may be tapered in a longitudinal direction, a circumferential direction, or in both such directions, in order to provide a variable rigidity (such as decreasing in thickness in a radial direction, width in a circumferential direction, or both, from a proximal end to a distal end thereof). The material of the body and the strain relief members may be the same.

The body may include a plug for receiving the catheter shaft. The plurality of strain relief members may have an arcuate shape in a circumferential direction for engaging an outer surface of the shaft. The hub may further include a packaging hoop in engagement with an external surface of the plurality of strain relief members.

According to another aspect of the disclosure, a catheter hub includes a body adapted for connecting to the catheter shaft, the body including a plurality of circumferentially spaced, elongated strain relief members having a decreasing rigidity from a proximal end to a distal end.

In one embodiment, the body includes a first port for supplying an inflation fluid to the catheter shaft and a second port for introducing a guidewire to the catheter shaft. The circumferentially spaced, elongated strain relief members may be tapered in a longitudinal direction, a circumferential direction, or in both such directions, and may be formed of the same material as the body.

The body may include a plug for receiving the shaft. The plurality of strain relief members may have an arcuate shape in a circumferential direction. A packaging hoop may also be provided in engagement with an external surface of the plurality of strain relief members.

According to a further aspect of the disclosure, an apparatus for performing a medical procedure includes a catheter shaft and a hub bonded to the catheter shaft. The hub includes a plurality of circumferentially spaced, elongated strain relief members of variable rigidity for engaging the catheter shaft.

In one embodiment, the body includes a first port for supplying an inflation fluid to the catheter shaft and a second port for introducing a guidewire to the catheter shaft. The circumferentially spaced, elongated strain relief members may be tapered in a longitudinal direction, a circumferential direction, or both such directions. The material of the plurality of strain relief members and the material of the body may be the same. In one version, the plurality of strain relief members have a decreasing rigidity from a proximal end to a distal end.

The catheter shaft may be bonded to the hub by an adhesive. The hub may include a plug for receiving the shaft. The strain relief members may have an arcuate shape in a circumferential direction.

Still a further aspect of the disclosure relates to a method of manufacturing a catheter hub. The method comprises attaching a plurality of elongated strain relief members of variable rigidity to the catheter hub. In one example, the attaching step comprises overmolding the strain relief members to a body of the catheter hub. The attaching step may comprise attaching the plurality of elongated strain relief members so as to be circumferentially spaced apart.

### BRIEF DESCRIPTION OF THE DRAWINGS

The above and further advantages of the disclosure may be better understood by referring to the following description in conjunction with the accompanying drawings in which:

FIG. 1 illustrates a side view of a balloon catheter including a hub at a proximal end thereof and a balloon at a distal end thereof.

FIG. 2 is a side view isolating the hub with enhanced strain relief according to the disclosure.

FIGS. 2A, 2B, and 2C are various views of the hub of FIG. 2.

FIG. 3 is a view of the hub including a packaging material.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the present invention. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

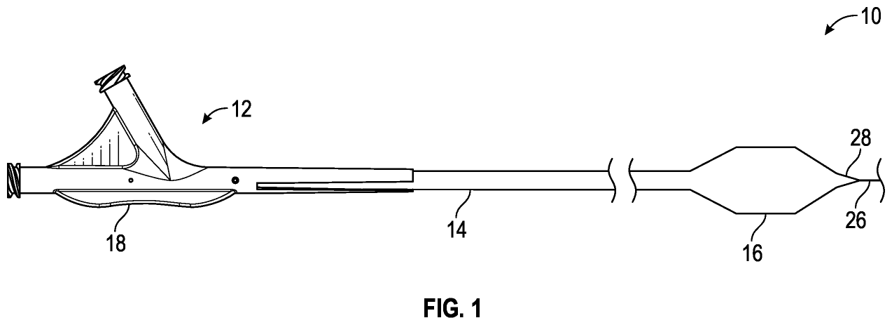

With reference to FIG. 1, a catheter 10 is shown, which includes a hub 12 adapted for providing enhanced strain relief for a catheter shaft 14. The hub 12 may be located as a proximal end of the shaft 14. The distal end of the shaft 14 may carry a working implement, such as an expandable element in the form of an inflatable balloon 16 adapted for performing percutaneous transluminal angioplasty (PTA).

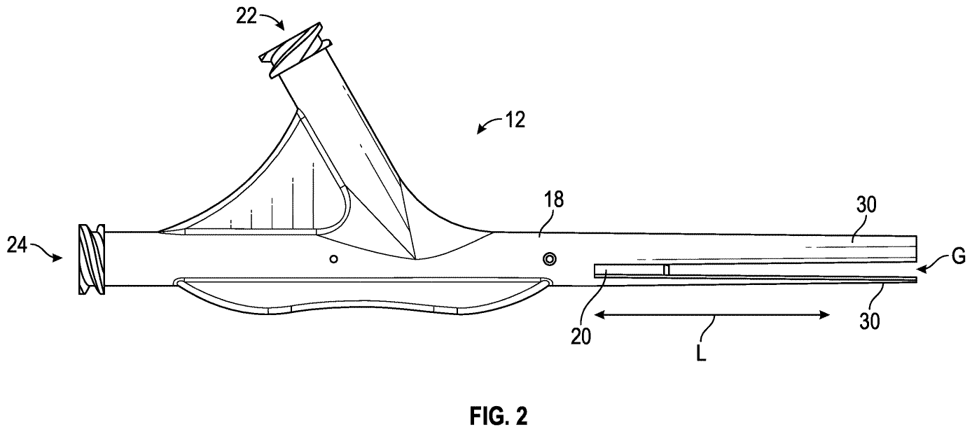

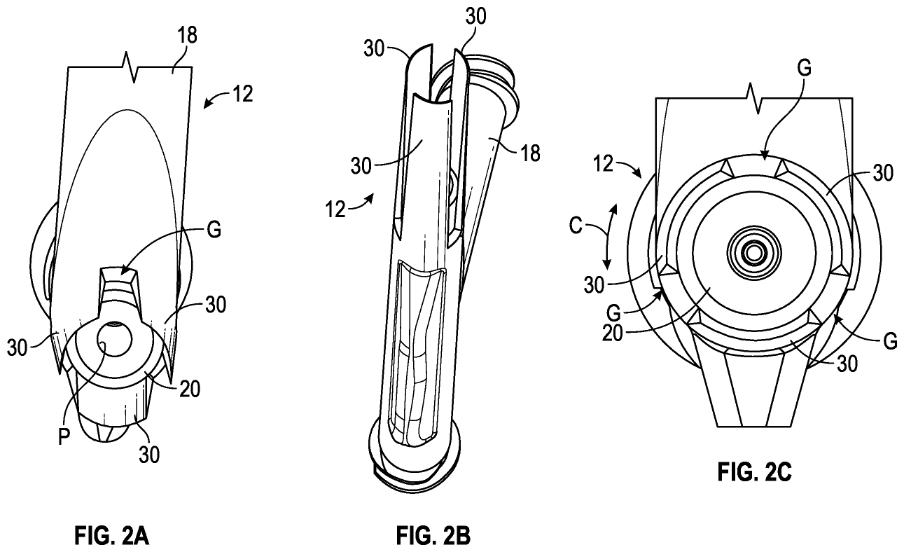

As perhaps best understood with reference to FIG. 2, the hub 12 comprises a body 18 adapted for connecting to the catheter shaft 14. The body 18 of the hub 12 may be attached to the catheter shaft 14 by bonding, such as an adhesive, weld, or mechanical connection. The body 18 may further include a plug 20 adapted to receive the proximal end of the shaft 14 and form a fluid-tight seal.

The body 18 may include a first port 22 for supplying an inflation fluid to the catheter shaft 14, such as via a passage P in the plug 20, and a second port 24 for introducing a guidewire 26 to the catheter shaft 12. The ports 22, 24 may also be associated with suitable connectors, such as luer connectors, to establish connections with external structures, such as for example an introducer or indeflator (not shown). The guidewire 26 may pass externally through a hollow tip 28 distal of the balloon 16. In view of the geometry of the exemplary arrangement shown, the hub 12 is often referred to as a “Y-hub,” but this disclosure is not limited to a hub having any particular shape, size, or geometry.

The catheter shaft 12 may also include a corresponding inflation lumen and guidewire lumen (not shown) communicating with the respective ports 22, 24. These lumens may be in a coaxial or side-by-side arrangement. A rapid-exchange arrangement could also be used, in which case the guidewire port 22 need not be present in the hub 12, and instead would be provided adjacent to a proximal end of the balloon 16, in which case the body 18 includes only inflation port 22.

With added reference to FIGS. 2A, 2B, and 2C, it can be understood that the body 18 includes a plurality of elongated strain relief members. In the illustrated version, three such members 30 are provided, but fewer or more may be present. These strain relief members 30 are spaced apart in a circumferential direction (note gaps G).

As can be appreciated, the strain relief members 30 have a curved or arcuate shape in cross section, and thus are adapted for contacting the outer surface of the shaft 14 to provide the desired strain relief. The members 30 may also continuously taper along longitudinal direction L, such as in width in the circumferential direction, thickness in the radial direction, or both width and thickness, in order to provide greater flexibility at a distal end than at a proximal end. Specifically, the members 30 may proceed longitudinally L from a wider proximal portion to a narrower distal portion, which may form an edge as shown in FIG. 2C. Likewise, the members 30 in a circumferential direction C may have a wider extent at a proximal end than at a distal end.

The members 30 may be at least partially formed of a material at least as rigid as the material of the body 18. For example, the material of both the body 18 and the members 30 may be inelastic, and have a modulus of elasticity, or Young's Modulus, of between about 1 and about 5 GPa, more specifically, from about 2 GPa to about 4 GPa, or still more specifically about 2.4 GPa. This is contrasted with the modulus of elasticity/Young's Modulus of a soft elastic material, such as silicone rubber, which typically ranges from 0.001 to 0.05 GPa.

In one particular example, one or more of the members 30 is formed so as to have a decreasing rigidity from a proximal end adjacent to the point of connection to the body 18 to a distal end spaced farthest from the point of connection with the body. In other words, the members 30 become more flexible in a longitudinal direction, which may be due to material property changes, thickness, or tapering. The body 18 including the members 30 may comprise a homogenous material, such as polyester, co-polyester, polycarbonate, polyethylene, polystyrene or polypropylene, but other or differential materials could also be used to achieve the desired benefits of strain relief for the catheter shaft 14.

The hub 18 including the strain relief members 30 may be manufactured by molding, such as injection molding. In one example, the hub 18 may be formed by over-molding the members 30 in a second mold after the body 18 is formed using a first mold, in order to form a unitary component. When the same material is used for forming the body 18 and relief members 30, this avoids the need for using a different material from that of the hub, such as silicone rubber, and adhesive to attach a separately formed strain relief member, thus reducing the manufacturing complexity and cost.

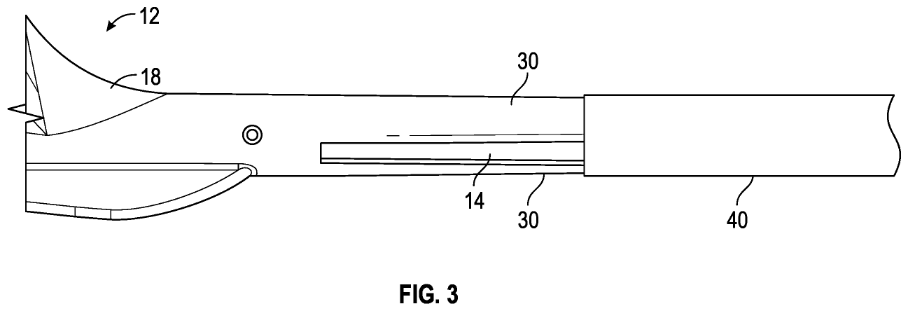

Turning to FIG. 3, it can also be understood that the strain relief members 30 may also aid in retaining a packaging structure for the catheter 10, such as a tubular packaging hoop 40, which serves to cover and protect the shaft 14 and associated balloon (in a folded state) following manufacturing and prior to use. Specifically, the inner surface of the packaging hoop 40 may engage an exterior surface of the members 30s, which as a result of friction helps to retain the hoop in place during transport and prior to use. This engagement is then overcome by simply sliding the hoop 40 in the distal direction to remove it from the catheter 10, which would then be ready for use in performing a medical procedure.

Summarizing, this disclosure relates to the following items:
1. A catheter hub providing enhanced strain relief for a catheter shaft, comprising:
   a body adapted for connecting to the catheter shaft, the body including a plurality of circumferentially spaced, elongated strain relief members formed of a material at least as rigid as a material of the body.
2. The catheter hub of item 1, wherein the body includes a first port for supplying an inflation fluid to the catheter shaft and a second port for introducing a guidewire to the catheter shaft.
3. The catheter hub of item 1 or item 2, wherein a thickness in a radial direction of each of the plurality of strain relief members tapers as these members extend in a longitudinal direction.
4. The catheter hub of any of items 1-3, wherein a width in a circumferential direction of the plurality of strain relief members tapers as these members extend in a longitudinal direction.
5. The catheter hub of any of items 1-4, wherein the material of the plurality of circumferentially spaced, elongated strain relief members and the material of the body are the same.
6. The catheter hub of any of items 1-5, wherein the plurality of circumferentially spaced, elongated strain relief members have a decreasing rigidity from a proximal end to a distal end.
7. The catheter hub of any of items 1-6, wherein the body includes a plug for receiving the catheter shaft.
8. The catheter hub of any of items 1-7, wherein the plurality of strain relief members have an arcuate shape in a circumferential direction.
9. The catheter hub of any of items 1-8, further including a packing hoop in engagement with an external surface of the plurality of strain relief members.
10. A catheter hub providing enhanced strain relief for a catheter shaft, comprising:
   a body adapted for connecting to the catheter shaft, the body including a plurality of circumferentially spaced, elongated strain relief members having a decreasing rigidity from a proximal end to a distal end.
11. The catheter hub of item 10, wherein the body includes a first port for supplying an inflation fluid to the catheter shaft and a second port for introducing a guidewire to the catheter shaft.
12. The catheter hub of item 10 or item 11, wherein a thickness in a radial direction of each of the plurality of strain relief members tapers as these members extend in a longitudinal direction.
13. The catheter hub of any of items 10-12, wherein a width in a circumferential direction of the plurality of strain relief members tapers as these members extend in a longitudinal direction.
14. The catheter hub of any of items 10-13 wherein the material of the plurality of circumferentially spaced, elongated strain relief members and the material of the body are the same.
15. The catheter hub of any of items 10-14, wherein the body includes a plug for receiving the shaft.
16. The catheter hub of any of items 10-15, wherein the plurality of strain relief members have an arcuate shape in a circumferential direction.
17. The catheter hub of any of items 10-16, further including a packing hoop in engagement with an external surface of the plurality of strain relief members.
18. An apparatus for performing a medical procedure, comprising:
   a catheter shaft; and
   a hub bonded to the catheter shaft, the hub including a plurality of circumferentially spaced, elongated strain relief members of variable rigidity for engaging the catheter shaft.
19. The apparatus of item 18, wherein the body includes a first port for supplying an inflation fluid to the catheter shaft and a second port for introducing a guidewire to the catheter shaft.
20. The apparatus of item 18 or item 19, wherein the material of the plurality of strain relief members and the material of the body are the same.
21. The apparatus of any of items 18-20, wherein the plurality of strain relief members have a decreasing rigidity from a proximal end to a distal end.
22. The apparatus of any of items 18-21, wherein the catheter shaft is bonded to the hub by an adhesive.
23. The apparatus of any of items 18-22, wherein the plurality of circumferentially spaced, elongated strain relief members are tapered in a longitudinal direction.
24. The apparatus of any of items 18-23, wherein the plurality of circumferentially spaced, elongated strain relief members are tapered in a circumferential direction.
25. The apparatus of any of items 18-24, wherein the body includes a plug for receiving the shaft.
26. The apparatus of any of items 18-25, wherein the plurality of strain relief members have an arcuate shape in a circumferential direction.
27. The apparatus of any of items 18-26, further including a packing hoop in engagement with an external surface of the plurality of strain relief members.
28. A method of manufacturing a catheter hub, comprising:
   attaching to the catheter hub a plurality of elongated strain relief members of variable rigidity adapted for engaging a catheter shaft.
29. The method of item 28, wherein the attaching step comprises overmolding the strain relief members to a body of the catheter hub.
30. The method of item 28 or item 29, wherein the attaching step comprises attaching the plurality of elongated strain relief members so as to be circumferentially spaced apart.

As used herein, the following terms have the following meanings:
“A”, “an”, and “the” as used herein refers to both singular and plural referents unless the context clearly dictates otherwise. By way of example, “a compartment” refers to one or more than one compartment.
“About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed.
“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A catheter hub for providing enhanced strain relief for a catheter shaft, comprising:
a body adapted for connecting to the catheter shaft, the body including a plurality of circumferentially spaced, elongated strain relief members formed of a material at least as rigid as a material of the body.

2. The catheter hub of claim 1, wherein the body includes a first port for supplying an inflation fluid to the catheter shaft and a second port for introducing a guidewire to the catheter shaft.

3. The catheter hub of claim 1, wherein a thickness in a radial direction of each of the plurality of strain relief members tapers as these members extend in a longitudinal direction.

4. The catheter hub of claim 1, wherein a width in a circumferential direction of the plurality of strain relief members tapers as these members extend in a longitudinal direction.

5. The catheter hub of claim 1, wherein the material of the plurality of strain relief members and the material of the body are the same material.

6. The catheter hub of claim 1, wherein the plurality of strain relief members have a decreasing rigidity from a proximal end to a distal end.

7. The catheter hub of claim 1, wherein the body includes a plug for receiving the catheter shaft.

8. The catheter hub of claim 1, wherein the plurality of strain relief members have an arcuate shape in a circumferential direction.

9. The catheter hub of claim 1, further including a packing hoop in engagement with an external surface of the plurality of strain relief members.

10. A catheter hub for providing enhanced strain relief for a catheter shaft, comprising:
a body adapted for connecting to the catheter shaft, the body including a plurality of circumferentially spaced, elongated strain relief members having a decreasing rigidity from a proximal end to a distal end.

11. The catheter hub of claim 10, wherein the body includes a first port for supplying an inflation fluid to the catheter shaft and a second port for introducing a guidewire to the catheter shaft.

12. The catheter hub of claim 10, wherein a thickness in a radial direction of each of the plurality of strain relief members tapers as these members extend in a longitudinal direction.

13. The catheter hub of claim 10, wherein a width in a circumferential direction of the plurality of strain relief members tapers as these members extend in a longitudinal direction.

14. The catheter hub of claim 10, wherein the material of the plurality of strain relief members and the material of the body are the same.

15. The catheter hub of claim 10, wherein the body includes a plug for receiving the shaft.

16. The catheter hub of claim 10, wherein the plurality of strain relief members have an arcuate shape in a circumferential direction.

17. The catheter hub of claim 10, further including a packing hoop in engagement with an external surface of the plurality of strain relief members.

18. An apparatus for performing a medical procedure, comprising:
a catheter shaft; and
a hub bonded to the catheter shaft, the hub including a plurality of circumferentially spaced, elongated strain relief members of variable rigidity for engaging the catheter shaft.

19. The apparatus of claim 18, wherein the body includes a first port for supplying an inflation fluid to the catheter shaft and a second port for introducing a guidewire to the catheter shaft.

20. The apparatus of claim 18, wherein the material of the plurality of strain relief members and the material of the body are the same.

21-30. (canceled)
